(19) United States
(12) Patent Application Publication
(10) Pub. No.: US 2016/0031790 A1
(43) Pub. Date: Feb. 4, 2016

(54) METHOD OF MANUFACTURE OF OCTANEDIOIC ACID, PRECURSORS, AND DERIVATIVES

(71) Applicant: SEGETIS, INC., Golden Valley, MN (US)

(72) Inventors: ERICH J. MOLITOR, ELK RIVER, MN (US); BRIAN D. MULLEN, DELANO, MN (US)

(21) Appl. No.: 14/777,069

(22) PCT Filed: Mar. 11, 2014
(86) PCT No.: PCT/US2014/023111

§ 371 Date: Sep. 15, 2015

Related U.S. Application Data
(60) Provisional application No. 61/790,826, filed on Mar. 15, 2013.

(51) Int. Cl. C07C 51/353; C07C 67/03; C07D 307/33; C07C 51/09; C07C 51/36; C07C 29/149; C07C 253/06; C07C 209/48; C07C 209/16; C07C 263/10; C07C 227/08; C07C 67/313; C07D 211/02; C07D 223/10; C07C 67/465

(52) U.S. Cl. CPC C07C 51/353; C07C 67/465; C07C 67/03; C07D 307/33; C07C 51/09; C07C 51/36; C07C 29/149; C07C 253/06; C07C 209/48; C07C 209/16; C07C 263/10; C07C 227/08; C07C 67/313; C07D 211/02; C07D 223/10

(57) ABSTRACT

A method for the manufacture of 1,8-octanedioic acid comprises: reacting gamma-valerolactone with an alcohol in the presence of an acid or a base catalyst to provide an alkyl pentenoate, converting the alkyl pentenoate in the presence of a metathesis initiator to provide the dialkyl octenedioate, reacting the dialkyl octenedioate with hydrogen in the presence of a hydrogenation catalyst to provide a dialkyl 1,8-octanedioate and hydrolyzing the dialkyl 1,8-octanedioate to provide the 1,8-octanedioic acid.

### PRIORITY

This application claims the priority to U.S. Provisional Patent Application Ser. No. 61/790,826, filed on Mar. 15, 2013, the contents of which are incorporated herein by reference in their entirety.

### BACKGROUND

This disclosure relates to a method for the manufacture of octanedioic acid, its precursors, and the derivatives of octanedioic acid and its precursors. These compounds can be used directly, or as intermediates to produce other derivatives.

### SUMMARY

A method for the manufacture of a dialkyl octenedioate having the formula (1)

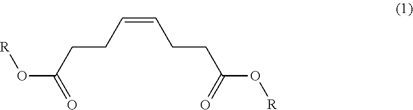

wherein R is a C₁₋₁₈ alkyl, preferably a C₁₋₁₂ alkyl, comprises: reacting gamma-valerolactone having the formula (2)

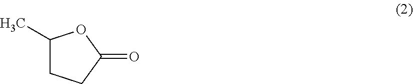

with an alcohol having the formula (3) R—OH (3) in the presence of an acid or a base catalyst to provide an alkyl pentenoate having the formula (4)

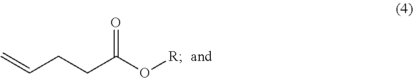

and
converting the alkyl pentenoate having the formula (4) in the presence of a metathesis initiator to provide the dialkyl octenedioate having the formula (1), wherein in formulas (3) and (4), R is a C₁₋₁₈ alkyl, preferably a C₁₋₁₂ alkyl.

The dialkyl octenedioate having the formula (1) can be converted to a dialkyl 1,8-octanedioate having the formula (10)

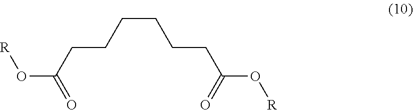

wherein R is a C₁₋₁₈ alkyl, preferably a C₁₋₁₂ alkyl; and the dialkyl 1,8-octanedioate having the formula (10) can be hydrolyzed to provide 1,8-octanedioic acid having the formula (9)

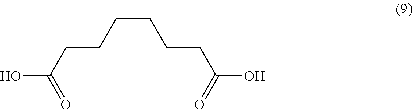 [SMILES: O=C(O)CCCCCCC(=O)O]

The dialkyl octenedioate having the formula (1) and the 1,8-octanedioic acid having the formula (9) can be used to prepare a variety of derivatives.

A compound produced by the above methods is also provided.

The above described and other embodiments are further described by the following detailed description and claims.

### DETAILED DESCRIPTION

Long chain linear aliphatic diacids are desirable for use in specialty polyamides, for example, nylon, and polyesters. However, these diacids can be expensive and difficult to obtain. For example, a member of this class, octanedioic acid, is currently prepared by oxidation of cyclooctene. Cyclooctene is a petrochemical derived material produced by butadiene dimerization followed by partial selective hydrogenation of cyclooctadiene. In practice, it would be desirable to avoid the oxidation chemistry. Further, there is an increasing demand for methods to produce chemicals from renewable sources to reduce the dependence on the fossil sources of carbon. Accordingly, there remains a need for a convenient and cost effective method for the manufacture of octanedioic acid, precursors, and derivatives thereof. It would be a further advantage if these materials can be derived from bio-sourced feedstocks.

Described herein is a method to produce octanedioic acid, its precursors, and derivatives that are otherwise difficult to obtain. As used herein, “octanedioic acid” refers to “1,8-octanedioic acid,” also known as “suberic acid.” An advantage of the method is that oxidation of cyclooctene is no longer needed. In a particularly advantageous feature, the starting material can be obtained from a bio-sourced feedstock, for example a carbohydrate. Furthermore, ethylene, which is widely used in the chemical industry, is a co-product in the process. In addition, a precursor of octanedioic acid, 4-octenedioate can be converted to a variety of derivatives by converting the double bond in the precursor to useful functional groups such as epoxides, diols, aldehydes, or esters.

### Precursors of Octanedioic Acid

A method for the manufacture of a dialkyl octenedioate of formula (1), a precursor of octanedioic acid, comprises reacting gamma-valerolactone of formula (2) with an alcohol of formula (3) to provide an alkyl pentenoate of formula (4); and converting the alkyl pentenoate of formula (4) in the presence of a metathesis initiator to provide the dialkyl octenedioate of formula (1). The method is illustrated in Scheme 1.

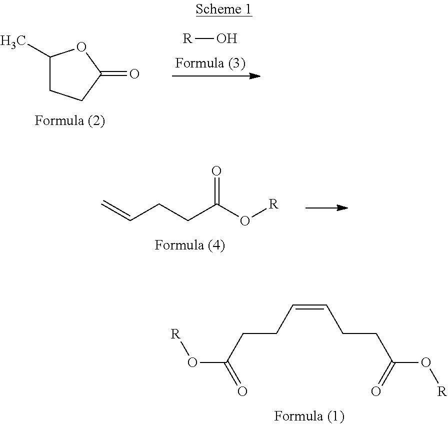

In formulas (1), (3) and (4), R is a C₁₋₁₈ alkyl, preferably a C₁₋₁₂ alkyl. Methyl is specifically mentioned.

The transesterification reaction between the gamma-valerolactone of formula (2) and the alcohol of formula (3) can be carried out at an elevated temperature, for example 50° C.-500° C., in the presence of an acid or a base catalyst. Exemplary acid catalyst includes acidic oxides of elements of main groups III and IV and subgroups IV and VI of the periodic table, as well as protic and Lewis acids as described in U.S. Pat. No. 4,740,613. The acid catalyst can also be acidic zeolitic catalysts as described in U.S. Pat. No. 5,144,061. Exemplary base catalyst includes metal oxides, hydroxides, carbonates, silicates, phosphates, and aluminates as described in U.S. Pat. No. 6,835,849.

The alkyl pentenoate of formula (4) can be converted to dialkyl octenedioate of formula (1) under metathesis conditions. The reaction temperature can range from about −20° C. to about 600° C., specifically from about 0° C. to about 500° C., more specifically from about 35° C. to about 400° C. Pressure depends on the boiling point of the solvent used, for example, sufficient pressure may be used to maintain a solvent liquid phase and can range from about 0 to about 2000 psig. Reaction times are not critical, and can be from several minutes to 48 hours. The reactions are generally carried out in an inert atmosphere, for example nitrogen or argon.

The metathesis reaction can be carried out in the absence or in the presence of a solvent. The reaction can also be carried out in a carbon dioxide medium as described in U.S. Pat. No. 5,840,820. Examples of solvents for the reaction include organic, protic, or aqueous solvents that are inert under the reaction conditions, such as aromatic hydrocarbons, chlorinated hydrocarbons, ethers, aliphatic hydrocarbons, alcohols, water, or a combination comprising at least one of the foregoing. Specifically, solvents include benzene, toluene, p-xylene, methylene chloride, dichloroethane, dichlorobenzene, tetrahydrofuran, diethylether, pentane, methanol, ethanol, water, or mixtures thereof. More specifically, the solvent can be benzene, toluene, p-xylene, methylene chloride, dichloroethane, dichlorobenzene, tetrahydrofuran, diethylether, pentane, methanol, ethanol, or mixtures thereof.

The olefin metathesis reaction is carried out in the presence of a metathesis initiator. The metathesis initiator initiates the metathesis reaction, and may or may not be recovered at the completion of the reaction. The term “initiator” as used herein refers to both true initiators (i.e., wherein the initiator is not recoverable at the completion of the reaction) and metathesis catalysts (i.e., wherein the initiator is recoverable at the completion of the reaction). Metathesis initiators may be generally classified into three main categories; transition metal carbene metathesis initiators, transition metal salts in combination with an alkylating agent, and transition metal complexes capable of forming an active metal carbene by reaction with an olefin.

Transition metal carbene initiators include complexes which are prepared apart from the metathesis reaction process and which contain a metal carbene functionality. Exemplary transition metal carbene metathesis initiators include carbenes based on transition metals including ruthenium, molybdenum, tantalum, osmium, iridium, titanium, and tungsten carbenes as described in U.S. Pat. Nos. 5,312,940 and 5,342,909 to Grubbs et al.

Metathesis initiator systems comprising a transition metal salt in combination with an alkylating agent include, for example transition metal salts based on molybdenum, tungsten, titanium, zirconium, tantalum, and rhenium together with an alkylating agent, such as butyl lithium, alkyl magnesium halides, alkyl aluminum halides, and alkyl or phenyl tin compounds. An activator may also be included to further facilitate the generation of the active carbene moiety. Examples of activators include oxygen, alcohols such as methanol and ethanol, epoxides, hydro peroxide, and peroxides.

Transition metal complexes capable of forming an active metal carbene by reaction with one or more of the olefins employed in the reaction do not require the addition of an alkylating agent or an activator. Metathesis catalysts of this type include transition metal complexes of ruthenium, osmium, tungsten, and iridium as described in U.S. Pat. No. 5,840,820.

A method for the manufacture of oct-4-ene-1,8-dioic acid of formula (7), an alternative precursor for octanedioic acid, comprises reacting gamma-valerolactone of formula (2) with an alcohol of formula (3) to provide an alkyl pentenoate of formula (4), hydrolyzing the alkyl pentenoate of formula (4) to provide 4-pentenoic acid of formula (8), and converting 4-pentenoic acid of formula (8) in the presence of a metathesis initiator to provide oct-4-ene-1,8-dioic acid of formula (7). The method is illustrated in Scheme 2.

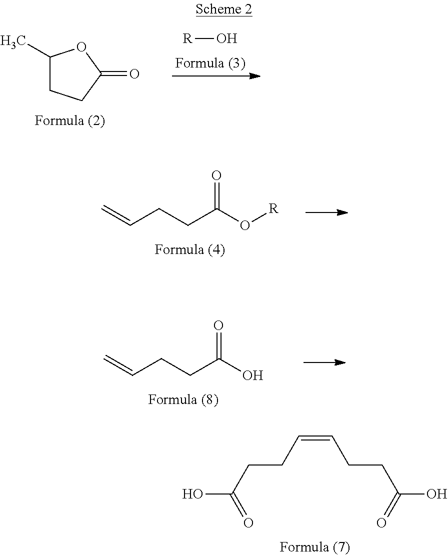

In formulas (3) and (4), R is a C₁₋₁₈ alkyl, preferably a C₁₋₁₂ alkyl.

The reaction conditions for the transesterification reaction between the gamma-valerolactone of formula (2) and the alcohol of formula (3) have been described herein. The transesterification product, alkyl pentenoate of formula (4), can then be hydrolyzed to provide 4-pentenoic acid of formula (8). To facilitate the reaction, the hydrolysis can be conducted at an elevated temperature in the presence of an acid or base catalyst. The formed 4-pentenoic acid of formula (8) can then be converted to oct-4-ene-1,8-dioic of formula (7) under metathesis conditions described herein.

Alternatively, the oct-4-ene-1,8-dioic acid of formula (7) can be derived from the dialkyl octenedioate of formula (1) by reacting with water in the presence of an acid or base catalyst. The reaction is illustrated in Scheme 3.

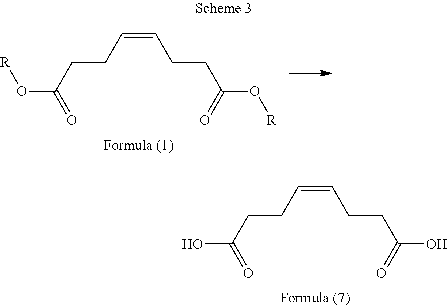

In formula (1), R is a C₁₋₁₈ alkyl, preferably a C₁₋₁₂ alkyl.

In a particularly advantageous feature, the starting material, gamma-valerolactone of formula (2) for the manufacture of dialkyl octenedioate of formula (1) and oct-4-ene-1,8-dioic acid of formula (7), can be obtained from a bio-sourced feedstock. Specifically, gamma-valerolactone of formula (2) can be derived from levulinic acid or a levulinic ester of formula (5) as shown in Scheme 4 or derived from angelica lactone of formula (6) as shown in Scheme 5. As used herein, “angelica lactone” means alpha-angelica lactone.

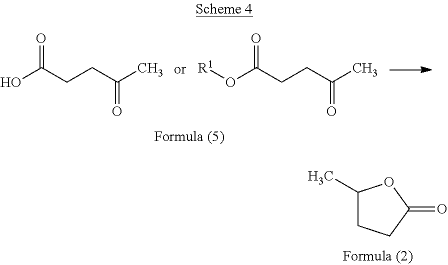

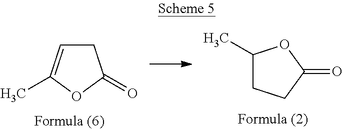

In formula (5), R¹ is a C₁₋₁₈ alkyl, preferably a C₁₋₁₂ alkyl

Levulinic acid is an abundant feedstock that is prepared on an industrial scale by acidic degradation of hexoses and hexose-containing polysaccharides such as cellulose, starch, sucrose, and the like. Levulinic acid and levulinic esters of formula (5) can be converted to gamma-valerolactone by catalytic hydrogenation. The conversion may proceed via hydrogenation to 4-hydroxy pentanoic acid followed by esterification to gamma-valerolactone. Processes for the conversion of levulinic acid into gamma-valerolactone are for example disclosed in U.S. Pat. No. 2,786,852, U.S. Pat. No. 4,420,622, U.S. Pat. No. 5,883,266, WO 02/074760 and WO 98/26869. A process for the catalytic hydrogenation of levulinate esters to form gamma-valerolactone is disclosed in EP 069409. An exemplary process for preparing gamma-valerolactone comprising heating levulinic acid in the presence of hydrogen and a catalytic amount of a metal catalyst, wherein the metal catalyst has both a hydrogenation and a ring-closing function, and wherein the metal catalyst is selected from the group consisting of Group VIII of the Periodic Table of Elements. Such catalysts are described in U.S. Pat. No. 6,617,464. Levulinic acid can also be reduced to gamma-valerolactone of formula (2) in the presence of ruthenium catalyst and formic acid as described in CN101376650.

Dehydration of levulinic acid provides angelica lactone of formula (6), which can in turn be hydrogenated to provide gamma-valerolactone of formula (2).

### Octanedioic Acid

A method for the manufacture of octanedioic acid of formula (9) comprises preparing the dialkyl octenedioate of formula (1) as described herein; converting the dialkyl octenedioate of formula (1) to a dialkyl 1,8-octanedioate of formula (10); and hydrolyzing the dialkyl 1,8-octanedioate of formula (10) to provide octanedioic acid of formula (9). The method is illustrated in Scheme 6.

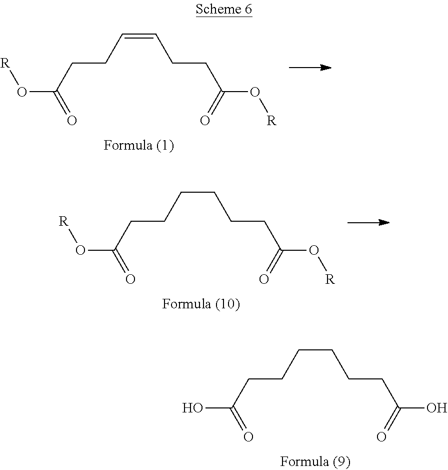

In formulas (1) and (10), R is a C₁₋₁₈ alkyl, preferably a C₁₋₁₂ alkyl

The dialkyl octenedioate of formula (1) can be converted to a dialkyl 1,8-octanedioate of formula (10) under hydrogenation conditions. Hydrogenation can be carried out in the presence of a catalyst, which can comprise a metal hydrogenation component deposited on a porous support material. The metal hydrogenation component comprises one or more metals for example nickel, platinum, palladium, rhodium, ruthenium, or a combination comprising at least one of the foregoing.

Alternatively, 1,8-dioctanedioic acid of formula (9) can be made by preparing oct-4-ene-1,8-dioic acid of formula (7) as described herein, and then converting the oct-4-ene-1,8-dioic acid of formula (7) to 1,8-dioctanedioic acid of formula (9) under hydrogenation conditions, for example in the presence of a supported or unsupported catalyst such as nickel, platinum, palladium, rhodium, ruthenium. The method is illustrated in Scheme 7.

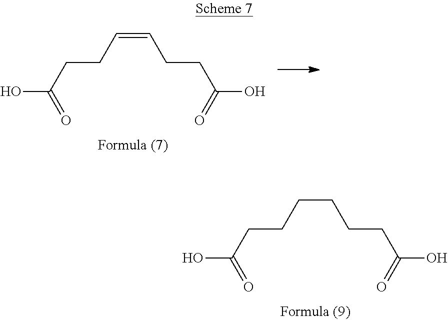 [SMILES: O=C(O)CC/C=C\CCC(=O)O ; O=C(O)CCCCCCC(=O)O]

### Derivatives of Octanedioic Acid and Octanedioic Acid Precursors

Derivatives of octanedioic acid and octanedioic acid precursors can also be manufactured and the methods will be discussed in detail hereinafter.

A method for the manufacture of 1,8-octane diol of formula (11) comprises preparing a dialkyl octenedioate of formula (1) according to the method described herein, and converting the dialkyl octenedioate of formula (1) to provide the 1,8-octane diol of formula (11). The method is illustrated in Scheme 8.

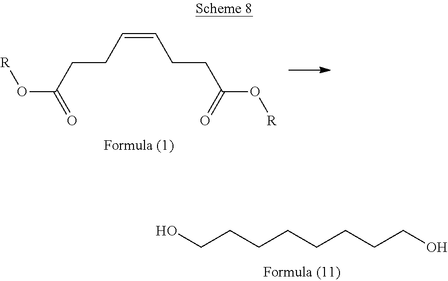

In formula (1), R is C₁₋₁₈ alkyl, preferably a C₁₋₁₂ alkyl.

The conversion of the dialkyl octenedioate of formula (1) to the 1,8-octane diol of formula (11) can be carried out by a hydrogenation process as described, for example, in U.S. Pat. No. 8,143,438. The catalytic hydrogenation can also be carried out using Cp*Ru complexes bearing a protic amine ligand as described in J. Am. Chem. Soc., 2011, 133 (12), pp. 4240-4242.

Alternatively, a method for the manufacture of 1,8-octane diol of formula (11) comprises preparing octanedioic acid having the formula (9) according to the method described herein; and then converting the octanedioic acid having the formula (9) to 1,8-octane diol of formula (11). The method is illustrated in Scheme 9.

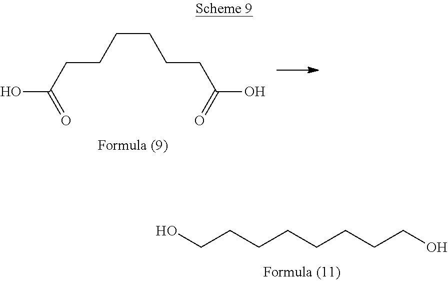 [SMILES: O=C(O)CCCCCCC(=O)O ; OCCCCCCCCO]

The conversion can be carried out in the presence of hydrogen and a hydrogenation catalyst. Exemplary catalyst includes titania supported platinum catalysts as described in Chem. Commun., 2010, 46, 6279-6281, copper, cobalt, and ruthenium catalysts as described, for example, in J. Am. Chem. Soc., 1955, 77 (14), pp. 3766-3768, U.S. Pat. No. 4,480,115 and U.S. Pat. No. 7,615,671.

A method for the manufacture of 1,6-dicyanohexane of formula (12) comprises preparing a dialkyl octenedioate of formula (1) according to the method described herein, converting the dialkyl octenedioate of formula (1) to a dialkyl 1,8-octanedioate of formula (10) by hydrogenation in the presence of a hydrogenation catalyst such as nickel, platinum, palladium, rhodium, ruthenium or a combination comprising at least one of the foregoing, and converting the dialkyl octanedioate of formula (10) to 1,6-dicyanohexane of formula (12), for example, by treating the dialkyl octanedioate of formula (10) with dimethylaluminum amide as described in Tetrahedron Letters (January 1979), 20 (51), pg. 4907-4910. The reaction is illustrated in Scheme 10.

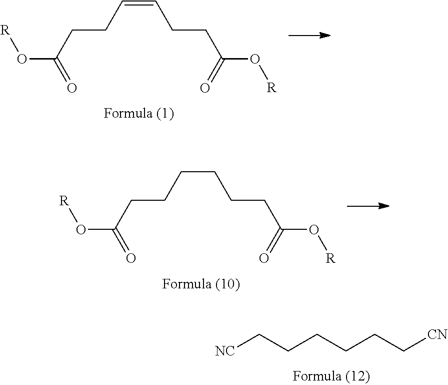

In formulas (1) and (10), R is C₁₋₁₈ alkyl, preferably a C₁₋₁₂ alkyl.

A method for the manufacture of 1,8-octane diamine of formula (13) comprises preparing 1,6-dicyanohexane of formula (12) according to the method described herein, then converting 1,6-dicyanohexane of formula (12) to 1,8-octane diamine of formula (13). The method is illustrated in Scheme 11.

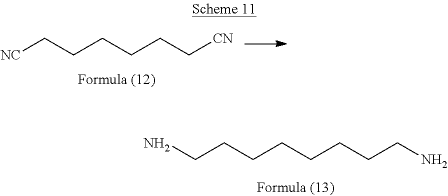 [SMILES: N#CCCCCCCC#N ; NCCCCCCCCN]

1,6-dicyanohexane of formula (12) can be reduced by reaction with hydrogen gas in the presence of ammonia and a metal catalyst such as cobalt, palladium, platinum, or nickel catalysts to provide 1,8-octane diamine of formula (14). The reaction can take place at an elevated temperature.

Alternatively, 1,8-octane diamine of formula (13) can be prepared from 1,8-octanediol of formula (11). The method comprises preparing 1,8-octanediol of formula (11) according to the method described herein, and converting 1,8-octanediol of formula (11) to 1,8-octane diamine of formula (13). The method is illustrated in Scheme 12.

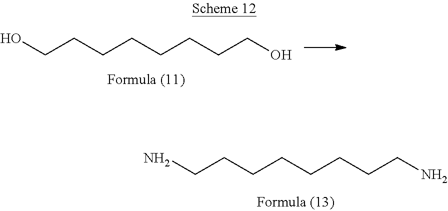 [SMILES: OCCCCCCCCO ; NCCCCCCCCN]

Converting 1,8-octanediol of formula (11) to 1,8-octane diamine of formula (13) comprises contacting 1,8-octanediol of formula (11) with ammonia and hydrogen in the presence of a catalyst. The contacting can be conducted at an elevated temperature and a superatmospheric pressure. The elevated temperature is above 50° C., above 75° C., above 100° C., or above 150° C. Specifically, the temperature is about 150° C. to about 350° C., more specifically about 175° C. to about 250° C. The superatmospheric pressure is above 100 kPa, above 500 kPa, above 1,000 kPa, above 5,000 kPa, above 10,000 kPa, or above 50,000 kPa. Specifically, the superatmospheric pressure is about 100 kPa to about 35,000 KPa, more specifically about 500 kPa to about 20,000 KPa.

The catalyst for use in the method can be a hydrogenation/dehydrogenation catalyst. In an embodiment, the catalyst comprises cobalt, nickel, copper, platinum, palladium, rhodium, ruthenium, rhenium, iron, chromium, oxides thereof, or a combination comprising at least one of the foregoing metal or metal oxide. In another embodiment, the catalyst comprises about 50 wt % to about 90 wt % of nickel, about 10 wt % to about 50 wt % of copper, and about 0.5 wt % to about 5 wt % of an oxide selected from chromium oxide, iron oxide, titanium oxide, thorium oxide, zirconium oxide, manganese oxide, magnesium oxide, zinc oxide, or a combination comprising at least one of the foregoing oxide. Such a catalyst can further comprise about 1 wt % to about 5-wt % of molybdenum. Similar catalysts are described in U.S. Pat. No. 5,530,127 and EP 0696572 and can be used. In another embodiment, the catalyst comprises a sponge-nickel catalyst. Other catalysts include supported metal catalysts, for example nickel or cobalt on silica or alumina (e.g., as described in U.S. Pat. No. 4,255,357 to Gardner et al., or U.S. Pat. No. 4,314,084 to Martinez et al.); zirconium oxide and nickel as described in WO 2008/000752: Cu/Ni/Zr/Sn catalysts as described in WO 2003/051508; bimetallic catalyst including nickel and rhenium supported on silica-alumina and also containing boron as described in U.S. Pat. No. 6,534,441; a catalyst comprising nickel, rhenium, cobalt, copper, and boron as described in U.S. Pat. No. 5,789,490; a catalyst comprising nickel, copper, and chromium as described in U.S. Pat. No. 2011/000970; bimetallic catalysts including 15 to 20 wt % nickel or cobalt and 0.5 to 3 wt % palladium on alumina, silica, or titania supports (e.g., as described in U.S. Pat. No. 5,932,769 to Vedage et al.); amorphous silica-alumina catalysts; metal-exchanged crystalline aluminosilicate catalysts (e.g., as described in U.S. Pat. No. 5,917,092); and zeolites, for example alkali metal modified mordenite, zeolite RHO, zeolite H-ZK-5, cobalt-exchanged Y-zeolite catalysts, and chabazite. The zeolites can be surface treated as described in U.S. Pat. No. 5,399,769 to F. C. Wilhelm et al., or silylated as described in U.S. Pat. No. 5,382,696 to T. Kiyoura et al.

A method for the manufacture of 1,8-octane diisocyanate of formula (14) comprises preparing 1,8-octane diamine of formula (13) according to the method described herein, and reacting 1,8-octane diamine of formula (13) with phosgene to provide 1,8-octane diisocyanate of formula (14). The method is illustrated in Scheme 13.

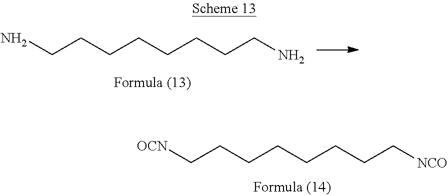

A method for the manufacture of a compound of formula (15) comprises preparing a dialkyl octenedioate of formula (1) according to the method described herein, reacting the dialkyl octenedioate with carbon monoxide and hydrogen to provide a compound of formula (16), and reacting the compound of formula (16) with an amine of formula (17) to provide the compound of formula (15). The method is illustrated in Scheme 14.

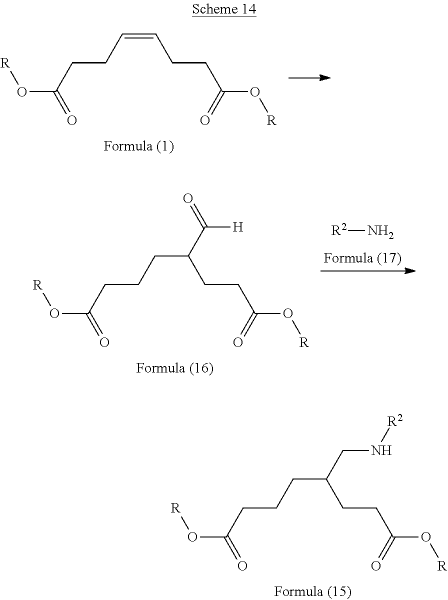

In formulas (1), (15), and (16), R is C₁₋₁₈ alkyl, preferably a C₁₋₁₂ alkyl. In formula (15), R² is hydrogen or C₁₋₁₈ alkyl, preferably a C₁₋₁₂ alkyl.

The dialkyl octenedioate of formula (1) can be converted to provide a compound of formula (16) by reacting with carbon monoxide and hydrogen, optionally in the presence of a suitable solvent, usually under superatmospheric pressure and in the presence of a catalyst, such as a transition metal carbonyl complex of rhodium or cobalt, for example octacarbonyldicobalt Co₂(CO)₈.

The compound of formula (16) can react with an amine or ammonia of formula (17) and hydrogen in the presence of a metallic hydrogenation catalyst to provide the compound of formula (15) as described in, for example, U.S. Pat. Nos. 20080167499; 6,046,359; 5,958,825; 20100222611; 20120116124; 7,230,134; and 4,152,353.

A method for the manufacture of a compound of formula (18), a compound of formula (19), or a combination comprising the compound of formula (18) and the compound of formula (19) comprises: preparing a compound of formula (16) according to the method described herein, cyclizing the compound of formula (16) to provide a mixture comprising the compound of formula (18), a compound of formula (19) and optionally separating the compound of formula (18) form the compound of formula (19). The method is illustrated in Scheme 15.

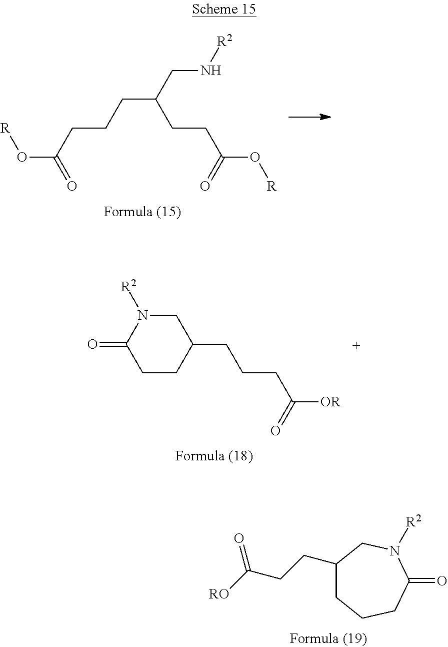

In formulas (15), (18), and (19), R is C₁₋₁₈ alkyl, preferably a C₁₋₁₂ alkyl, and R² is hydrogen or C₁₋₁₈ alkyl, preferably a C₁₋₁₂ alkyl.

Cyclizing comprises heating the compound of formula (15) at an elevated temperature optionally in the presence of a base. Illustratively, the compound of formula (15) can be heated in the presence of trimethylaluminum in hexane as described in Organic Syntheses, Coll. Vol. 6, p. 492 (1988); Vol. 59, p. 49 (1979).

A method for the manufacture of a compound of formula (34), a compound of formula (35), or a combination comprising the compound of formula (34) and the compound of formula (35) comprises: preparing a compound of formula (15) according to the method described herein, hydrolyzing the compound of formula (15) to provide an amino diacid of formula (20), cyclizing the amino diacid of formula (20) to provide a mixture comprising the compound of formula (34) and the compound of formula (35), and optionally separating the compound of formula (34) from the compound of formula (35). The method is illustrated in Scheme 16.

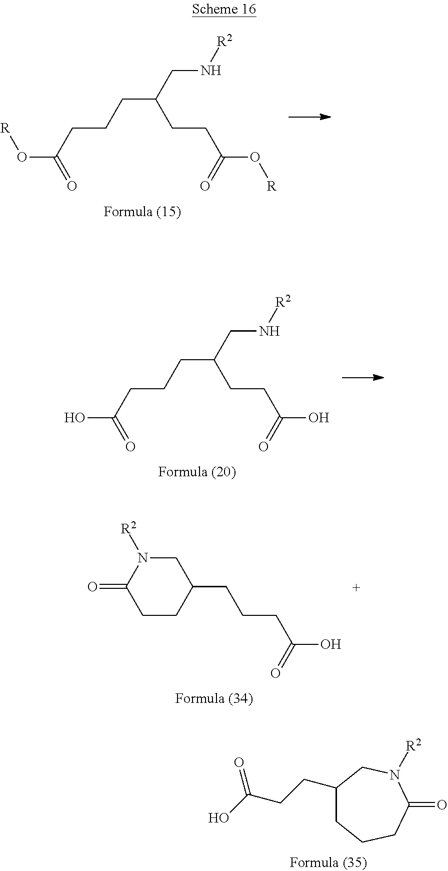

In formulas (15), (20), (34), and (35), R is C₁₋₁₈ alkyl, preferably a C₁₋₁₂ alkyl, and R² is hydrogen or C₁₋₁₈ alkyl, preferably a C₁₋₁₂ alkyl.

“Cyclizing the amino diacid of formula (20)” optionally comprises activating the carboxylic acid groups of the compound of formula (20), for example, by converting the acid groups to acyl halide groups.

A method for the manufacture of a triisocyanate of formula (21) comprises preparing a dialkyl octenedioate of formula (1) according to the method described herein, converting the dialkyl octenedioate of formula (1) to a compound of formula (16), for example, by reacting with carbon monoxide and hydrogen, optionally in the presence of a suitable solvent, usually under superatmospheric pressure and in the presence of a catalyst, such as a transition metal carbonyl complex of rhodium or cobalt, for example octacarbonyldicobalt Co₂(CO)₈, converting the compound of formula (16) to a triol having the formula (22) in the presence of hydrogen and a hydrogenation catalyst, converting the triol of formula (22) to a triamine of formula (23) under reductive amination conditions as described herein, and reacting the triamine of formula (23) with phosgene to provide the triisocyanate of formula (21). The method is illustrated in Scheme 17.

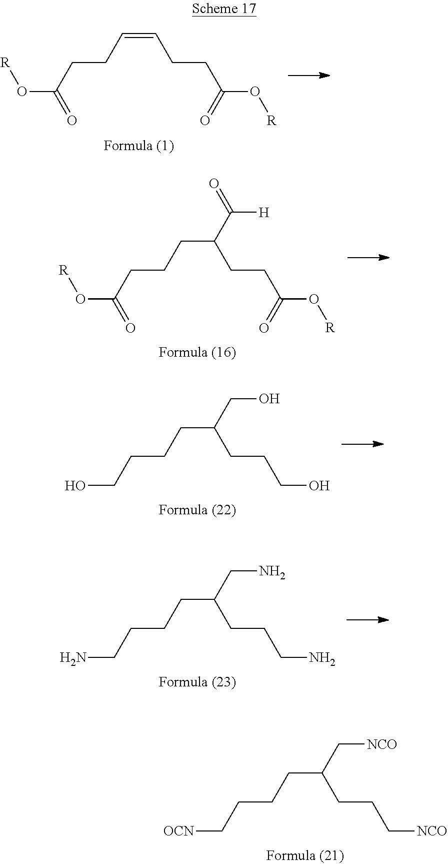

In formulas (1) and (16), R is C₁₋₁₈ alkyl, preferably a C₁₋₁₂ alkyl.

A method for the manufacture of a triester of formula (24) comprises preparing a dialkyl octenedioate of formula (1) according to the method described herein, converting the dialkyl octenedioate of formula (1) to the triester of formula (24) in the presence of carbon monoxide, R³OH, and a metal carbonyl catalyst under carbonylation conditions. The method is illustrated in Scheme 18.

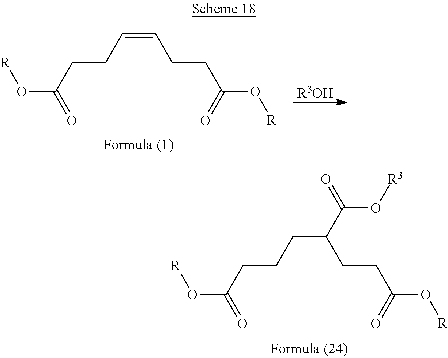

In formulas (1) and (24), R and R³ are C₁₋₁₈ alkyl, preferably a C₁₋₁₂ alkyl.

A method for the manufacture of a triol of formula (22) comprises preparing the triester of formula (24) as described herein, converting the triester of formula (24) to the triol of formula (22) by a hydrogenation process as described, for example, in U.S. Pat. No. 8,143,438. The catalytic hydrogenation can also be carried out using Cp*Ru complexes bearing a protic amine ligand as described in J. Am. Chem. Soc., 2011, 133 (12), pp. 4240-4242. The method is illustrated in Scheme 19.

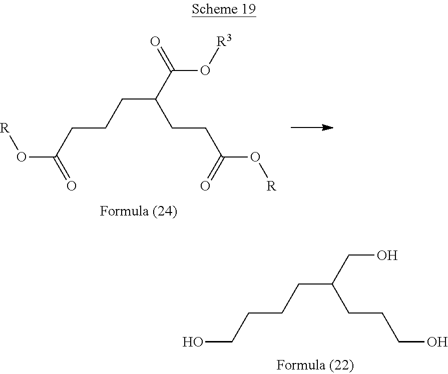

In formula (24), R and R³ are C₁₋₁₈ alkyl, preferably a C₁₋₁₂ alkyl.

A method for the manufacture of a triacid of formula (26) comprises preparing the triester of formula (24) as described herein, hydrolyzing the triester of formula (24) to provide the triacid of formula (26). The method is illustrated in Scheme 20.

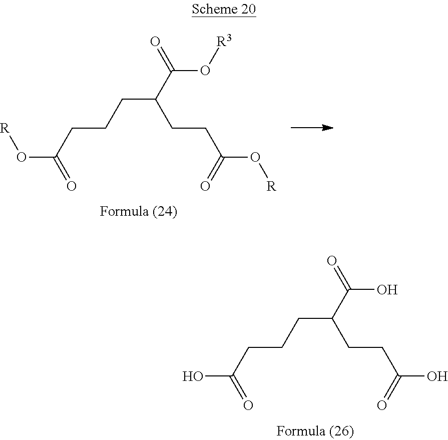

In formula (24), R and R³ are C₁₋₁₈ alkyl, preferably a C₁₋₁₂ alkyl.

A method for the manufacture of an epoxy-diester of formula (27) comprising preparing a dialkyl octenedioate of formula (1) with a peroxide-containing compound to provide the epoxy-diester of formula (27). The method is illustrated in Scheme 21.

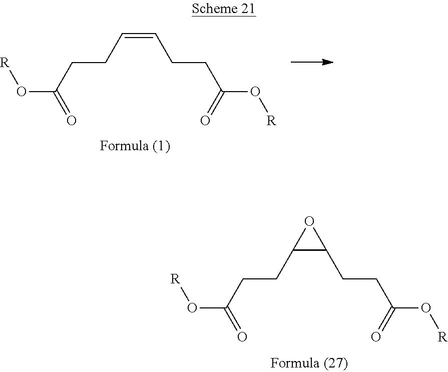

In formulas (1) and (27), R is C₁₋₁₈ alkyl, preferably a C₁₋₁₂ alkyl.

Peroxide-containing compounds include hydrogen peroxide, peroxycarboxylic acids (generated in-situ or preformed), and alkyl hydroperoxides. Other peroxide-containing reagents such as dimethyldioxirane can also be used.

A method for the manufacture of a ketal-triester of formula (28) comprises preparing the epoxy-diester of formula (27) according to the method described herein, reacting the epoxy-diester of formula (27) with a levulinic ester of formula (5) to provide the ketal-triester of formula (28). The method is illustrated in Scheme 22.

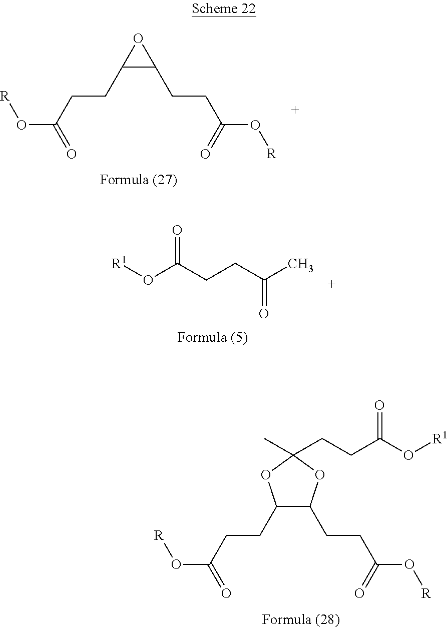

In formulas (5), (27) and (28), R and R¹ are C₁₋₁₈ alkyl, preferably a C₁₋₁₂ alkyl.

The reaction can be Bronsted or Lewis acid catalyzed as described, for example, in Journal of Organic Chemistry, 65(22), 7700-7702, 2000 for p-toluenesulfonic acid and in Organic Process Research & Development, 7 (3), 432-435, 2003 for using BF₃-Et₂O as a Lewis acid catalyst.

Alternatively, a method for the manufacture of a ketal-triester of formula (28) comprises preparing the epoxy-diester of formula (27) according to the method described herein, hydrolyzing the epoxy-diester of formula (27) to provide an epoxy-diacid of formula (29), converting the epoxy-diacid of formula (29) to a diacid-diol of formula (30), for example, by hydrolysis in the presence of an acid catalyst, converting the diacid-diol of formula (30) to the ketal-triester of formula (28) in the presence of a levulinic ester of formula (5) and an alcohol ROH. An acid catalyst can be used in the conversion from compound of formula (30) to the compound of formula (28). The method is illustrated in Scheme 23.

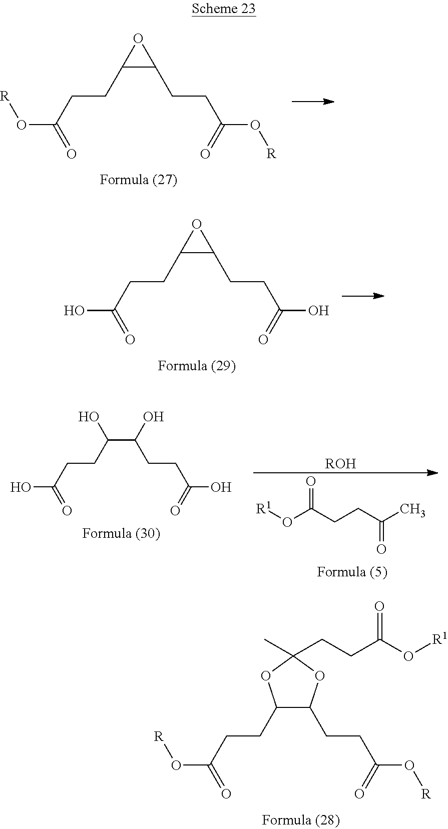

In formulas (5), (27) and (28), R and R¹ are C₁₋₁₈ alkyl, preferably a C₁₋₁₂ alkyl.

In another embodiment, a method for the manufacture of a ketal-triester of formula (28) comprises preparing the epoxy-diester of formula (27) according to the method described herein, converting the epoxy-diester of formula (27) to a diester-diol of formula (31), for example, by hydrolysis in the presence of an acid catalyst, reacting the diester-diol of formula (31) with a levulinic ester of formula (5) optionally in the presence of an acid catalyst, to provide the ketal-triester of formula (28). The method is illustrated in Scheme 24.

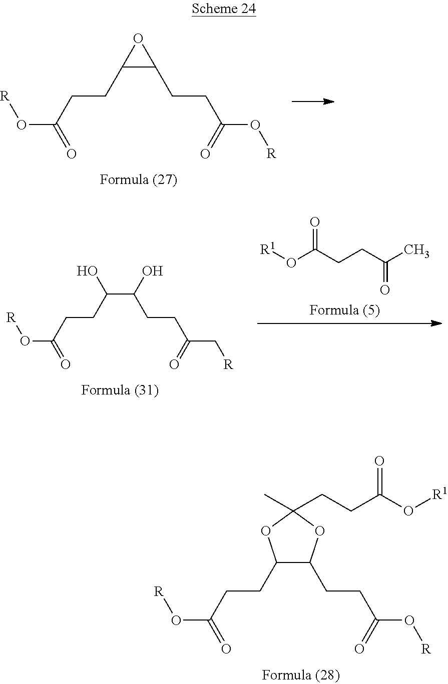

In formulas (5), (27), (28) and (31), R and R¹ are C₁₋₁₈ alkyl, preferably a C₁₋₁₂ alkyl.

A method for the manufacture of a bis-butyrolactone having the formula (32), a fused-bislactone having the formula (33), or a combination comprising the bis-butyrolactone and the fused-bislactone comprises preparing the epoxy-diester of formula (27) according to the method described herein, converting the epoxy-diester of formula (27) to a diester-diol of formula (31), converting the diester-diol of formula (31) to a mixture comprising the bis-butyrolactone of formula (32) and the fused-bislactone of formula (33) under esterification conditions, and optionally separating the bis-butyrolactone from the fused-bislactone. The method is illustrated in Scheme 25.

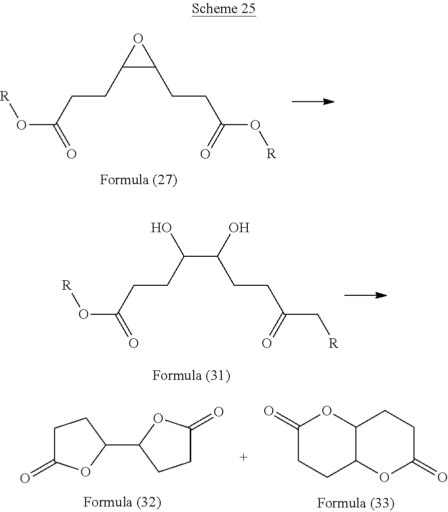

In formulas (27) and (31), R is C₁₋₁₈ alkyl, preferably a C₁₋₁₂ alkyl.

Alternatively, a method for the manufacture of a bis-butyrolactone having the formula (32), a fused-bislactone having the formula (33), or a combination comprising the bis-butyrolactone and the fused-bislactone comprises preparing the epoxy-diester of formula (27) according to the method described herein, converting the epoxy-diester of formula (27) to a diester-diol of formula (31), hydrolyzing the diester-diol of formula (31) to provide a diacid-diol of formula (30), converting the diacid-diol of formula (30) to a mixture comprising the bis-butyrolactone of formula (32) and the fused-bislactone of formula (33), and optionally separating the bis-butyrolactone from the fused-bislactone. The method is illustrated in Scheme 26.

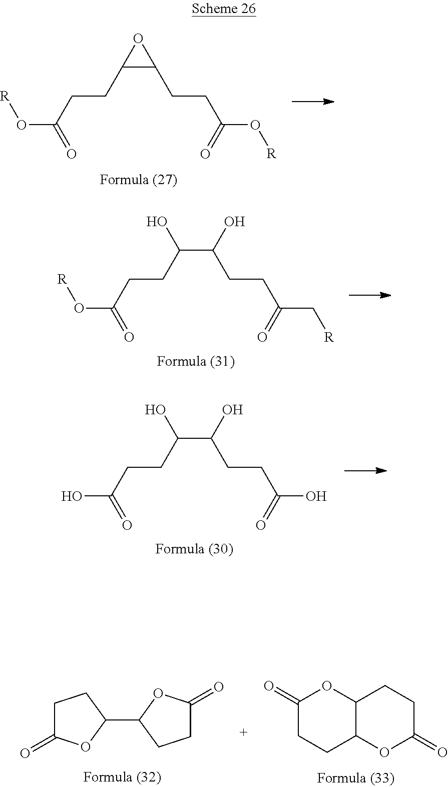

In formulas (27) and (31), R and R¹ are C₁₋₁₈ alkyl, preferably a C₁₋₁₂ alkyl.

In another embodiment, a method for the manufacture of a bis-butyrolactone having the formula (32), a fused-bislactone having the formula (33), or a combination comprising the bis-butyrolactone and the fused-bislactone comprises preparing the epoxy-diester of formula (27) according to the method described herein, hydrolyzing the epoxy-diester of formula (27) to provide an epoxy-diacid of formula (29), converting the epoxy-diacid of formula (29) to a diacid-diol of formula (30), converting the diacid-diol of formula (30) to a mixture comprising the bis-butyrolactone of formula (32) and the fused-bislactone of formula (33), and optionally separating the bis-butyrolactone from the fused-bislactone. The method is illustrated in Scheme 27.

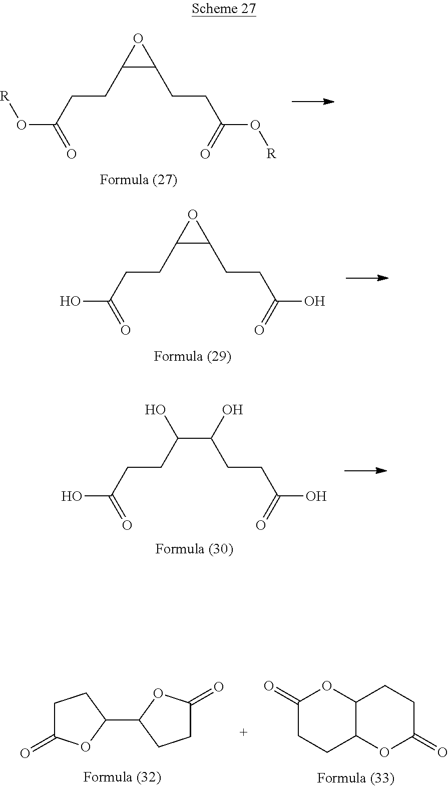

In formula (27), R is C₁₋₁₈ alkyl, preferably a C₁₋₁₂ alkyl.

### EXAMPLES

The following examples employed the following GC conditions:

GC: 7890A (Agilent Technolgies Inc.) FID detector
Column: Restek Rxi-5 ms
30 meter, 0.25 mm ID, 0.25 um film thickness
Inlet 250° C.
Split 25:1
Sample flow 2 mL/min (He carrier gas)
H₂ (30 mL/min; Air: 400 mL/min; He: 25 mL/min
Gradient 50° C. for 4 min, 20° C./min to 330, hold for 7 min

GC-MS: 7890C with 5975C MSD (Agilent Technoligies, Inc.)
Column: Restek Rxi-5 ms
30 meter, 0.25 mm ID, 0.25 um film thickness
MS Source (230); MS Quad (150)
Inlet 250° C.
Split 10:1
Sample flow 2 mL/min (He carrier gas)
Gradient 50° C. for 4 min, 20° C./min to 330, hold for 7 min

### Example 1

### Metathesis of Methyl Pentenoate to Yield Dimethyl Octenedioate Compound

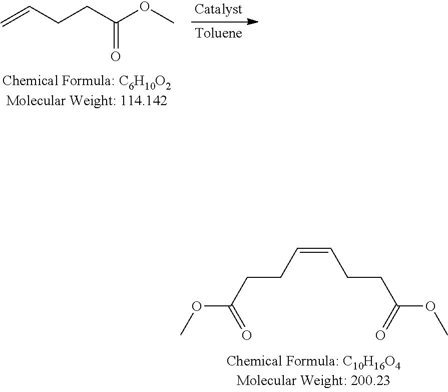

A sample of methyl 4-pentenoate in water and methanol solvent was assayed by GC/FID using an internal standard. The assay showed the sample contained 11.76 wt % of methyl 4-pentenoate (MP). The sample (18.23 g, 2.15 g MP) was combined with toluene (18.4 g) to provide a clear solution. The toluene solution was washed with 8 wt % sodium chloride solution (8.16 g). The bottom aqueous layer (22.02 g) was removed. The top organic layer was washed again with 8% sodium chloride solution (6.88 g), and 9.77 g was removed. The toluene layer (20.13 g) was assayed to be 9.68 wt % MP (1.95 g MP). The toluene solution was dried with MgSO₄ and then decanted and filtered (syringe filter, 0.45 μm, polypropylene) into a 250 mL 3-neck round bottom flask. The flask was magnetically stirred and purged with nitrogen for 30 minutes. Grubbs generation I catalyst (0.073 g) was added and the mixture was stirred at 23.5° C. After 17 hours, the reaction conversion was 65.8%. Additional catalyst (0.040 g) was added and stirring under nitrogen was continued until 65 hours. The product solution was concentrated by removal of toluene and the concentrated product solution (4.1 g) was saved for a subsequent reaction. Table 1 lists the final composition by GC analysis which showed 6.5% methyl 4-pentenoate, 6.19% of 7 carbon diester (m/z 186), and 80.9% desired product.

TABLE 1

| | Retention | |
|---|---|---|
| Compound | Time (min) | Area % |
| methyl 4-pentenoate | 4.27 | 6.53 |
| methyl cis-2-pentenoate | 4.50 | 0.457 |
| methyl 3-pentenoate (2 peaks) | 4.94 | 1.51 |
| methyl trans-2-pentenoate | 5.41 | 2.02 |
| Methyl hexenoate (2 isomers) | 6.31 | 1.25 |
| 3-Heptenedioic acid, 1,7-dimethyl ester (2 peaks) | 10.91 | 6.18 |
| 4-Octenedioic acid, 1,8-dimethyl ester (2 peaks) | 10.78 | 80.9 |

### Example 2

### Metathesis of Methyl Pentenoate to Yield Dimethyl Octenedioate Compound

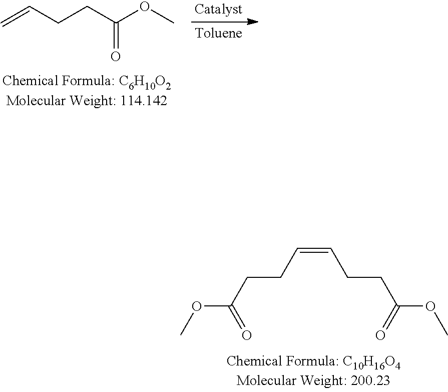

A sample of methyl 4-pentenoate and water/methanol (5.47 g) was combined with toluene (19.33 g) to form a clear solution. The solution was washed with 6.4 g of 8% brine solution. The phases were allowed to settle and 8.35 g of aqueous solution and 22.58 g of organic solution were collected. The organic toluene solution was assayed by GC/FID and was found to contain 14.43 wt % (3.26 g) of methyl 4-pentenoate (MP). The toluene solution was dried with MgSO₄ and then decanted and filtered (syringe filter, 0.45 μm, polypropylene) into a 250 mL 3-neck round bottom flask. The flask was magnetically stirred and purged with nitrogen for 30 minutes. Grubbs generation I catalyst (0.108 g) was added and the mixture was stirred at 24.2° C. Table 2 shows the product composition at various times. Table 3 shows the final; compositions. After 15 hours, the GC area % showed the product composition to be 19.3% methyl 4-pentenoate, 6.25% 3-heptenedioic acid, 1,7-dimethyl ester, and 68.8% 4-octenedioic acid, 1,8-dimethyl ester. The reaction was continued and heated to 35° C. for 5 hours and then at room temperature for 24 hours. The product solution (8.6 g) was collected.

TABLE 2

| Time | | | |
|---|---|---|---|
| (hr) | 4-methyl pentenoate | Dimethyl heptenoate | Dimethyl octenoate |
| 15 | 19.3 | 6.25 | 68.8 |
| 20 | 8.98 | 6.94 | 79.43 |
| 44 | 6.38 | 7.08 | 80.81 |

TABLE 3

| | Retention | |
|---|---|---|
| Compound | Time (min) | Area % |
| methyl 4-pentenoate | 4.27 | 6.38 |
| methyl cis-2-pentenoate | 4.5 | 0.49 |
| methyl 3-pentenoate (2 peaks) | 4.94 | 0.89 |
| methyl trans-2-pentenoate | 5.41 | 2.27 |
| Methyl hexenoate (2 isomers) | 6.31 | 1.35 |
| 3-Heptenedioic acid, 1,7-dimethyl ester (2 peaks) | 10.91 | 7.08 |
| 4-Octenedioic acid, 1,8-dimethyl ester (2 peaks) | 10.78 | 80.8 |

### Example 3

### Hydrogenation of Dimethyl Octenedioate to Yield Dimethyl Suberate

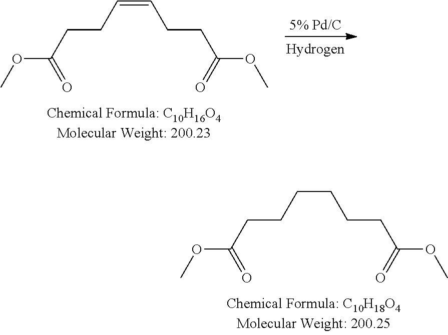

To a 1 L Parr reactor vessel was loaded 5% palladium on carbon catalyst (1.6 g, BASF ESCAT 147), methanol (91 g), and 4.08 g of product solution from example 1 (containing approximately 1.3 g of dimethyl octenedioate). The Parr vessel was sealed, purged with nitrogen (150 psig×3), and then pressurized with hydrogen (150 psig) and heated to 75° C. for 4 hours as stirred with a magnetic stirrer. Table 4 lists the final composition by GC analysis of the product solution which showed complete conversion to the saturated products.

TABLE 4

| Compound | Retention Time (min) | GC area % |
|---|---|---|
| Methyl pentanoate | 4.59 | 8.59 |
| Methyl hexanoate | 6.29 | 1.27 |
| Dimethyl heptandioate | 10.21 | 6.3 |
| Dimethyl octanedioate | 10.93 | 81.5 |

The product was filtered to remove catalyst using a syringe filter (0.45 u, polypropylene). The product and methanol solution was placed in a 250 mL round bottom flask and the methanol, toluene, and part of the methyl pentanoate were distilled out at atmospheric pressure to leave 1.16 g of liquid product.

### Example 4

### Metathesis of Pentenoic Acid to Yield Octenedioic Acid

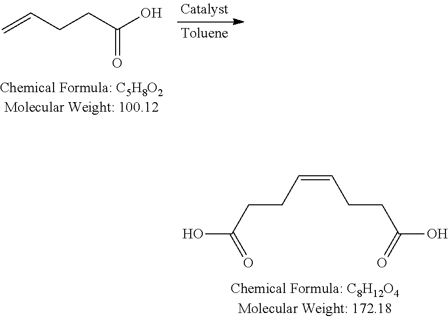

To a 250 mL round bottom flask was added 20 mL of HPLC grade toluene. The flask was purged with nitrogen and pentenoic acid (Aldrich, 4.89 g, 49.94 mmol) was added followed by 0.105 g of Grubbs generation I catalyst. The reaction was stirred under a slow nitrogen stream at room temperature (21-25° C.) for 66 hours. Additional catalyst was added during the reaction. The reaction mixture was analyzed by GC/FID and GC/MS analysis at various times as shown in table 5. Starting material and isomers accounted for 95% and diacid was present in 5%.

TABLE 5

| Total reaction time (hr) | Additional Catalyst (mg) | Conversion (GC %) |
|---|---|---|
| 23 | | 3 |
| 24 | 30 | |
| 46 | | 4 |
| 48 | 30 | |
| 66 | | 5 |

### Example 5

### Saponification of Dimethyl Suberate to Yield Suberic Acid

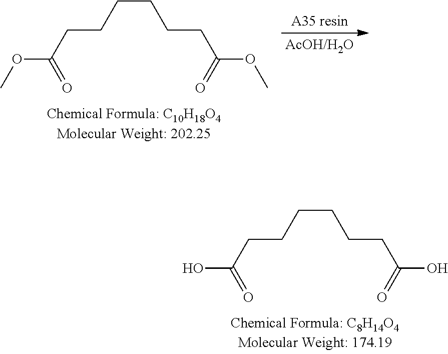

To a 250 mL round bottom flask equipped with a stir bar, heating mantle, temperature probe, and short path distillation head was added crude dimethyl suberate (1.15 g), Amberlyst 35 (0.2 g, pre-washed with methanol), acetic acid (10 mL), and DI water (2 mL). The reaction was heated to 95° C. for 20 hours with a gentle stream of nitrogen (0.05 SCFH) passing through the headspace. GC analysis showed high conversion of dimethyl suberate to suberic acid as shown in Table 6.

TABLE 6
GC analysis of crude reaction mixture

| Compound | Retention Time (min) | GC area % |
|---|---|---|
| Heptandioic acid | 10.77 | 5.85 |
| Dimethyl suberate | 10.88 | 0.04 |
| Monomethyl suberic acid | 11.11 | 2.02 |
| Suberic acid | 11.49 | 90.87 |

The product solution was cooled and filtered to remove catalyst using a syringe filter (0.45 u, polypropylene). The clear product solution was loaded to a 250 mL round bottom flask equipped with a stir bar, heating mantle, temperature probe, and short path distillation head. The acetic acid was distilled out under atmospheric pressure to leave an oily residue. To the residue was added DI water (10 mL). The water was distilled out to leave about 4 mL of solution which was allowed to gradually cool to room temperature with the stirring off. Upon cooling, white crystals were evident. The solid was isolated by filtration and washed with DI water (4 mL). The solids were dried at 100° C. to yield 0.41 g of white crystalline product. A sample of the product was dissolved in acetone for GC analysis and results are shown in Table 7.

TABLE 7
GC analysis of crystallized product

| Compound | Retention Time (min) | GC area % |
|---|---|---|
| Heptandioic acid | 10.77 | 0.34 |
| Dimethyl suberate | 10.88 | 0.147 |
| Monomethyl suberic acid | 11.11 | 10.6 |
| Suberic acid | 11.49 | 88.58 |

The singular forms “a,” “an,” and “the” include plural referents unless the context clearly dictates otherwise. “Or” means “and/or.” The endpoints of all ranges directed to the same component or property are inclusive and independently combinable. The suffix “(s)” as used herein is intended to include both the singular and the plural of the term that it modifies, thereby including at least one of that term (e.g., “colorant(s)” includes at least one colorant). “Optional” or “optionally” means that the subsequently described event or circumstance can or cannot occur, and that the description includes instances where the event occurs and instances where it does not. Unless defined otherwise, technical and scientific terms used herein have the same meaning as is commonly understood by one of skill in the art to which this invention belongs.

As used herein, a “combination” is inclusive of blends, mixtures, alloys, reaction products, and the like. Compounds are described using standard nomenclature. For example, any position not substituted by any indicated group is understood to have its valency filled by a bond as indicated, or a hydrogen atom.

As used herein, the term “alkyl” refers to a straight or branched chain, saturated monovalent hydrocarbon group.

All references cited herein are incorporated by reference in their entirety. While typical embodiments have been set forth for the purpose of illustration, the foregoing descriptions should not be deemed to be a limitation on the scope herein. Accordingly, various modifications, adaptations, and alternatives can occur to one skilled in the art without departing from the spirit and scope herein.

What is claimed is:

1. A method for the manufacture of a dialkyl octenedioate having the formula (1) 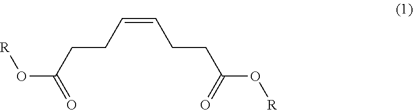
wherein R is a C₁₋₁₈ alkyl,
the method comprising:
reacting gamma-valerolactone having the formula (2) 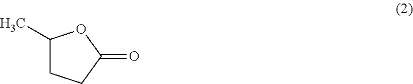
with an alcohol having the formula (3)
R—OH (3)
in the presence of an acid or a base catalyst to provide an alkyl pentenoate having the formula (4) 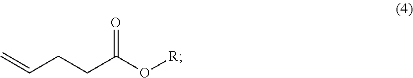
and
converting the alkyl pentenoate having the formula (4) in the presence of a metathesis initiator to provide the dialkyl octenedioate having the formula (1),
wherein in formulas (3) and (4), R is a C₁₋₁₈ alkyl.

2. The method of claim 1, wherein the metathesis initiator is a transition metal carbene metathesis initiator, a transition metal salt in combination with an alkylating agent, and a transition metal complex capable of forming an active metal carbene by reaction with an olefin.

3. The method of claim 1, wherein converting the alkyl pentenoate having the formula (4) to the dialkyl octenedioate having the formula (1) comprises conducting the metathesis at a temperature of about −20° C. to about 600° C. and a pressure of about 0 to about 2000 psig.

4. The method of claim 1, further comprising converting levulinic acid or a levulinic ester having the formula (5) 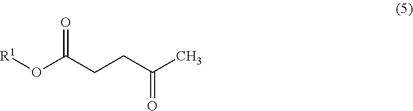
wherein R¹ is a C₁₋₁₈ alkyl, to gamma-valerolactone having the formula (2).

5. The method of claim 1, further comprising converting angelica lactone having the formula (6) 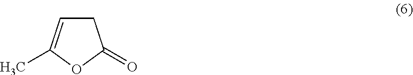
to gamma-valerolactone having the formula (2).

6. A method for the manufacture of oct-4-ene-1,8-dioic acid having the formula (7) 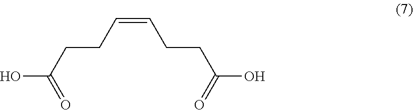 [SMILES: O=C(O)CC/C=C\CCC(=O)O]
the method comprising:
reacting gamma-valerolactone having the formula (2) 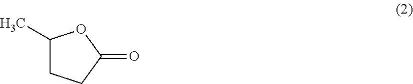
with an alcohol having the formula (3)
ROH (3)
in the presence of an acid or a base catalyst to provide an alkyl pentenoate having the formula (4) 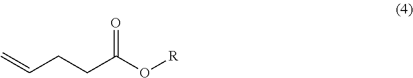
wherein R is a C₁₋₁₈ alkyl;
hydrolyzing the alkyl pentenoate having the formula (4) to provide 4-pentenoic acid having the formula (8) 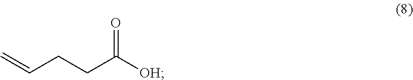
and
converting 4-pentenoic acid having the formula (8) in the presence of a metathesis catalyst to provide oct-4-ene-1,8-dioic acid having the formula (7).

7. A method for the manufacture of oct-4-ene-1,8-dioic acid having the formula (7) 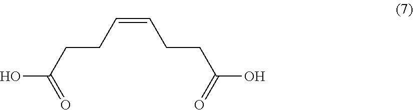 [SMILES: O=C(O)CC/C=C\CCC(=O)O]
the method comprising:
preparing a dialkyl octenedioate having the formula (1) 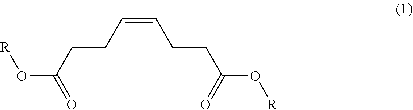
according to claim 1; and
hydrolyzing the dialkyl octenedioate having the formula (1) to provide oct-4-ene-1,8-dioic acid having the formula (7).

8. A method for the manufacture of 1,8-octanedioic acid having the formula (9) 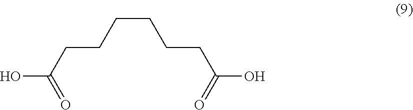 [SMILES: O=C(O)CCCCCCC(=O)O]
the method comprising:
preparing a dialkyl octenedioate having the formula (1) according to claim 1 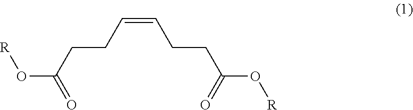
wherein R is C₁₋₁₈ alkyl;
converting the dialkyl octenedioate having the formula (1) to a dialkyl 1,8-octanedioate having the formula (10) 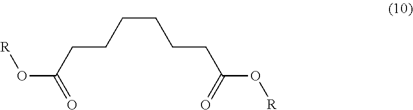
wherein R is a C₁₋₁₈ alkyl; and
hydrolyzing the dialkyl 1,8-octanedioate having the formula (10) to provide 1,8-octanedioic acid having the formula (9).

9. A method for the manufacture of 1,8-octanedioic acid having the formula (9) 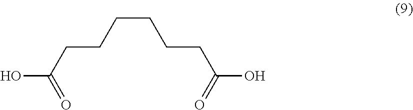 [SMILES: O=C(O)CCCCCCC(=O)O]
the method comprising:
preparing oct-4-ene-1,8-dioic acid having the formula (7) according to claim 6 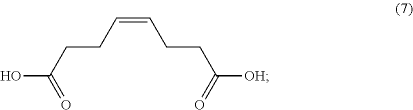 [SMILES: O=C(O)CC/C=C\CCC(=O)O]
and
converting oct-4-ene-1,8-dioic acid having the formula (7) to 1,8-octanedioic acid having the formula (9).

10. A method for the manufacture of 1,8-octane diol having the formula (11) 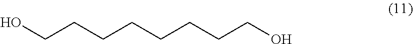
the method comprising:
preparing a dialkyl octenedioate having the formula (1) according to claim 1 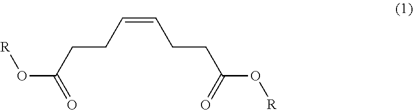
wherein R is a C₁₋₁₈ alkyl; and
converting the dialkyl octenedioate having the formula (1) to 1,8-octane diol having the formula (11).

11. A method for the manufacture of 1,8-octane diol having the formula (11) 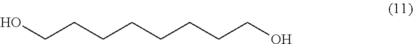
the method comprising:
preparing 1,8-octanedioic acid having the formula (9) according to claim 8 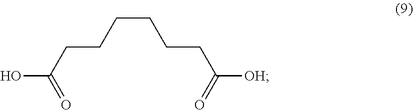 [SMILES: O=C(O)CCCCCCC(=O)O]
and
converting the 1,8-octanedioic acid having the formula (9) to 1,8-octane diol having the formula (11).

12. A method for the manufacture of 1,6-dicyanohexane having the formula (12) 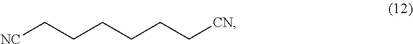
the method comprising:
preparing a dialkyl octenedioate having the formula (1) according to claim 1 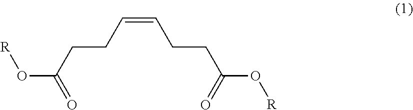
wherein R is a C₁₋₁₈ alkyl;
reacting the dialkyl octenedioate having the formula (1) with hydrogen under hydrogenation conditions to provide a dialkyl 1,8-octanedioate having the formula (10) 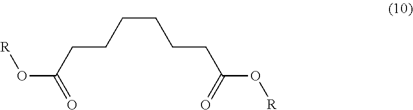
wherein R is a C₁₋₁₈ alkyl; and
converting the dialkyl octanedioate having the formula (10) to 1,6-dicyanohexane having the formula (12).

13. A method for the manufacture of 1,8-octane diamine having the formula (13) 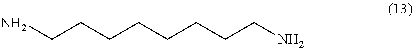
the method comprising:
preparing 1,6-dicyanohexane having the formula (12) according to claim 12 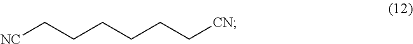
and
reacting 1,6-dicyanohexane having the formula (12) with hydrogen in the presence of a nickel or cobalt catalyst to provide the 1,8-octane diamine having the formula (14).

14. A method for the manufacture of 1,8-octane diamine having the formula (13) 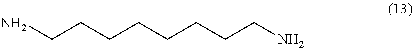
the method comprising:
preparing 1,8-octanediol having the formula (11) according to claim 10 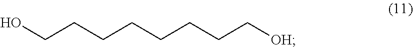
and
converting 1,8-octanediol having the formula (11) to 1,8-octane diamine having the formula (13).

15. A method for the manufacture of 1,8-octane diisocyanate having the formula (14) 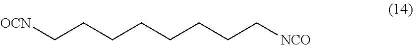
the method comprising:
preparing 1,8-octane diamine having the formula (13) according to claim 13 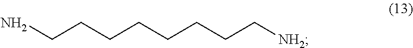
and
reacting the 1,8-octane diamine having the formula (13) with phosgene to provide the 1,8-octane diisocyanate having the formula (14).

16. A method for the manufacture of a compound of the formula (15) 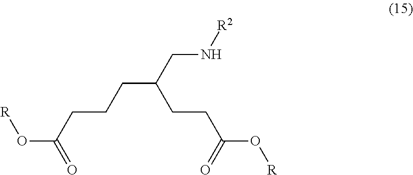
wherein R is C₁₋₁₈ alkyl, R² is H or C₁₋₁₈ alkyl,
the method comprising:
preparing a dialkyl octenedioate having the formula (1) according to claim 1 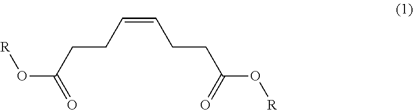
R is C₁₋₁₈ alkyl;
reacting the dialkyl octenedioate with carbon monoxide and hydrogen to provide a compound of the formula (16) 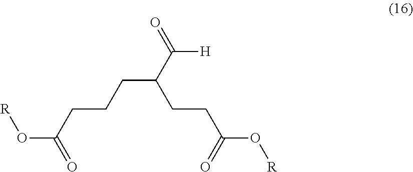
wherein R is C₁₋₁₈ alkyl; and
reacting the compound of the formula (16) with an amine of the formula (17)
R²—NH₂ (17)
wherein R² is H or C₁₋₁₈ alkyl, to provide the compound of formula (15).

17. A method for the manufacture of a compound having the formula (18), a compound having the formula (19), or a combination comprising the compound having the formula (18) and the compound having the formula (19) 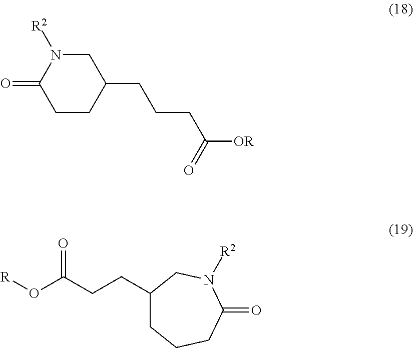
wherein in formulas (18) and (19), R is a C₁₋₁₈ alkyl; and R² is C₁₋₁₈ alkyl or H;
the method comprising:
preparing a compound having the formula (15) according to claim 16 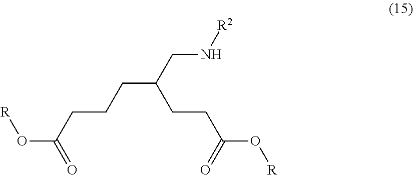
wherein R is C₁₋₁₈ alkyl, R² is hydrogen or C₁₋₁₈ alkyl;
cyclizing the compound having the formula (15) to provide a mixture comprising the compound having the formula (18) and a compound having the formula (19); and
optionally separating the compound having the formula (18) from the compound having the formula (19).

18. A method for the manufacture of a compound having the formula (34) and a compound having the formula (35), or a combination comprising the compound having the formula (34) and the compound having the formula (35) 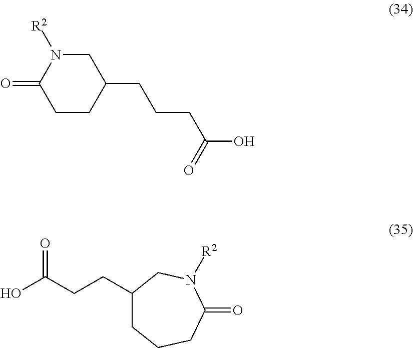
the method comprising:
preparing a compound having the formula (15) according to claim 16 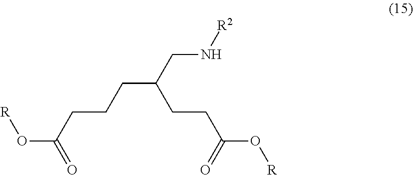
wherein R is a C₁₋₁₈ alkyl, R² is C₁₋₁₈ alkyl or hydrogen;
converting the compound having the formula (15) to an amino diacid having the formula (20) 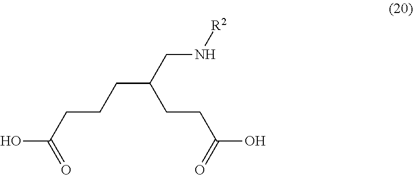
cyclizing the amino diacid having the formula (20) to provide a mixture comprising the compound having the formula (34) and a compound having the formula (35); and
optionally separating the compound having the formula (34) from the compound having the formula (35).

19. A method for the manufacture of a triisocyanate having the formula (21) 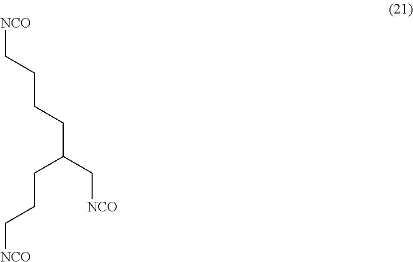
the method comprising:
preparing a dialkyl octenedioate having the formula (1) according to claim 1;
converting the dialkyl octenedioate having the formula (1) to a compound having the formula (16), 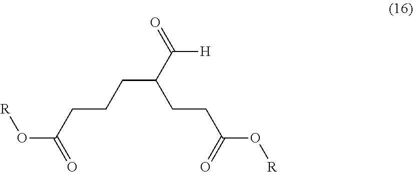
wherein R is C₁₋₁₈ alkyl;
converting the compound of the formula (16) to a triol having the formula (22) 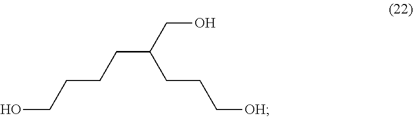 [SMILES: OCCCCC(CO)CCCO]
converting the triol having the formula (22) to a triamine having the formula (23) 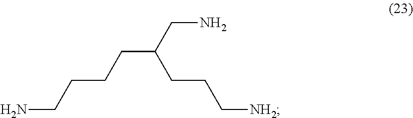 [SMILES: NCCCCC(CN)CCCN]
and
reacting the triamine having the formula (23) with phosgene to provide the triisocyanate of the formula (21).

20. A method for the manufacture of a triester having the formula (24) 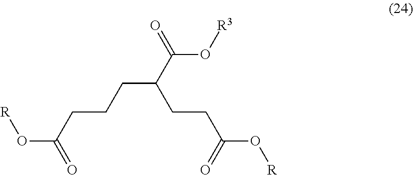
wherein R and R³ are independently C₁₋₁₈ alkyl,
the method comprising:
preparing a dialkyl octenedioate having the formula (1) according to claim 1 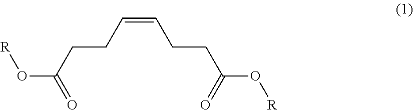
R is C₁₋₁₈ alkyl; and
converting the dialkyl octenedioate having the formula (1) to the triester having the formula (24) in the presence of carbon monoxide, R³OH wherein R³ is a C₁₋₁₈ alkyl, and a catalyst under carbonylation conditions.

21.-32. (canceled)
